(19) United States
(12) Patent Application Publication
(10) Pub. No.: US 2011/0293556 A1
(43) Pub. Date: Dec. 1, 2011

(54) TREATMENT OF INSULIN RESISTANCE/METABOLIC SYNDROME TO ALLEVIATE THE RISKS OF DEMENTIA

(75) Inventors: Stuart A. LIPTON, Rancho Santa Fe, CA (US); Daniel EINHORN, La Jolla, CA (US)

(73) Assignee: SANFORD-BURNHAM MEDICAL RESEARCH INSTITUTE, La Jolla, CA (US)

(21) Appl. No.: 13/205,534

(22) Filed: Aug. 8, 2011

Related U.S. Application Data
(63) Continuation of application No. 11/569,063, filed on Jan. 28, 2008, now abandoned.
(60) Provisional application No. 60/569,724, filed on May 10, 2004.

(51) Int. Cl. A61K 38/19; A61K 38/22; A61K 31/137; A61K 31/13; A61K 31/5685; A61B 5/00; A61K 31/64; A61K 31/155; A61K 31/426; A61K 31/5585; A61P 25/00; A61P 3/08; A61K 38/28; A61K 31/455

(52) U.S. Cl. 424/85.1; 514/5.9; 514/6.8; 514/654; 514/611; 514/178; 514/356; 514/592; 514/635; 514/369; 514/469; 600/301

(57) ABSTRACT

This invention relates to Applicant's discovery that Metabolic Syndrome , a cluster of disorders stemming from a resistance to insulin, contributes directly to dementia, particularly Alzheimer's disease. Applicant's invention includes a screening method to determine susceptibility and diagnosis of dementia based on the risk factors for Metabolic Syndrome. Applicant's invention further includes methods for the prevention or treatment of dementia and other neurological conditions based on (1) minimizing insulin resistance, thereby preventing excess biosynthesis of insulin; (2) modulating the activity of IDE such that insulin competes less efficiently with β-amyloid protein for the TDE; and (3) blocking the consequences of NMDA receptor activation, such as by minimizing the generation of NO and other harmful free radicals.

### RELATED APPLICATION

Benefit of priority under 35 U.S.C. 119(e) is claimed herein to U.S. Provisional Application No.: 60/569,724, filed May 10, 2004. The disclosure of the above referenced application is incorporated by reference in its entirety herein.

### FIELD OF THE INVENTION

This invention is directed to methods of treatment of insulin resistance or metabolic syndrome in order to alleviate the risks of dementia, in view of the newly discovered link between the occurrence of insulin resistance or metabolic syndrome.

### BACKGROUND OF THE INVENTION

Dementia is an age related syndrome, the incidence of which is predicted to double every six-years of life expectancy. Alzheimer's disease (AD) is the most frequent form of dementia; vascular dementia (VaD) being probably somewhat less frequent. The initial stages of dementia are characterized by problems in cognition and some functional impairment. Common pathological hallmarks in AD are the neurofibrillary tangles and senile plaques, in which a major component is beta amyloid protein in the plaque and hyperphosphorylated tau protein in the tangle.

Current treatments for AD target the cholinergic system (symptomatic treatment) and inhibition of the pathologically enhanced glutamatergic activity (neuroprotective treatment). In the central nervous system, glutamate and gamma-aminobutyric acid (GABA) are the major excitatory and inhibitory neurotransmitters, respectively. Glutamate activates several types of ionotropic receptors, including N-methyl-D-aspartate (NMDA) receptors.

The NMDA receptor has unique properties distinguishing it from the other glutamate receptor subtypes. First, the activation of NMDA receptor requires the presence of dual agonists, glutamate and glycine. The ligand-gated ion channel of the NMDA receptor is, thus, under the control of at least two distinct allosteric sites. In addition, the NMDA receptor controls the flow of both divalent (Ca²⁺) and monovalent (Na⁺, K⁺) ions into the postsynaptic neural cell through a receptor associated channel. (Foster et al., “Taking apart NMDA receptors”, Nature, 329:395-396, 1987; Mayer et al., “Excitatory amino acid receptors, second messengers and regulation of intracellular Ca²⁺ in mammalian neurons,” Trends in Pharmacol. Sci., 11:254-260, 1990). The activation of these receptors is regulated by Mg²⁺ in a voltage-dependent manner (i.e., the NMDA receptor is blocked at resting membrane potential and activated when depolarized). Most importantly; however, the NMDA receptor is extremely permeable to Ca²⁺, a key regulator of cell function.

NMDARs are believed to play a pivotal role in the transmission of excitatory signals from primary sensory neurons to the brain through the spinal cord (A. H. Dickenson (1990) Trends Pharmacol. Sci., 11, 307-309) as well as many other types of neurons intrinsic to the brain and important in learning and memory, development, and plasticity (S. A. Lipton and P. A. Rosenberg (1994) N. Engl J. Med., 330, 613-622). NMDA receptors mediate Ca²⁺ influx into neurons, and its receptor-gated channel activity is blocked by M²⁺ in a voltage-dependent manner. These unique properties allow NMDA receptors to play a critical role in development of the nervous system, synaptic plasticity, memory, and other physiological processes in the CNS.

However, excessive stimulation of NMDA receptors has also been implicated in many pathological conditions including chronic neurodegenerative states, such as Alzheimer's disease, Huntington's disease, HIV-associated dementia, Parkinson's disease, multiple sclerosis, amyotrophic lateral sclerosis (ALS), and glaucoma. Prolonged activation of glutamate NMDA receptors leads to excessive Ca²⁺ influx to the cell, via an overexpression of NMDA receptors at the cell surface. This excitotoxic state is known to contribute to AD.

Thus there is a need in the art to further understand the mechanism behind this increased expression of NMDA receptors at the surface of a cell, thus allowing for the proper diagnosing and treatment of the conditions causing said event. There is a further need in the art to develop compounds that are pharmaceutically active in modulating the expression of NMDA receptors at the cell membrane.

### BRIEF SUMMARY OF THE INVENTION

The current invention is related to Applicant's discovery that Metabolic Syndrome, a cluster of disorders stemming from a resistance to insulin, contributes directly to dementia, particularly Alzheimer's disease. Sleep and mood disorders accompanying insulin resistance can aggravate symptoms of neurological degenerative disorders. Applicant's invention includes a screening method to determine susceptibility and diagnosis of dementia based on the risk factors for Metabolic Syndrome (hereinafter “Insulin Resistance”). Dementia is aggravated by sleep and mood disorders. Applicant's invention further includes prevention and treatment of dementia with therapeutic compounds commonly used for preventing and treating other abnormalities associated with Insulin Resistance. Applicant's discovery further includes developing novel therapeutic compounds useful for the prevention and treatment of dementia associated with Insulin Resistance.

### DETAILED DESCRIPTION OF THE INVENTION

Applicant has discovered that individuals presenting with the risk factors associated with Insulin Resistance are at an increased risk for developing dementia, thereby leading to a screening method to determine those at risk of developing dementia. The risk factors for developing Insulin Resistance are well documented. The American Association of Clinical Endocrinologists (AACE) and the American College of Endocrinology (ACE) recognizes the following factors as some of the indicators of risk for developing one or more of the cluster of disorders associated with Insulin Resistance: A waistline of 40 inches or greater for men or 35 inches or greater for women as measured across the belly; a body mass index greater than 25 kg/m²; a blood pressure of 130/85 mm Hg or more; a triglyceride level above 150 milligrams per deciliter; a fasting blood glucose level greater than 110 milligrams per deciliter; and a high density lipoprotein (HDL) level less than 40 milligrams per deciliter for men or less than 50 milligrams per deciliter for women. The most sensitive currently available factor is the blood glucose level 2 hours after a 75 gram oral glucose challenge; a level above 140 milligrams per deciliter is abnormal. Applicant's discovery that those presenting with one or more, preferably three or more, of these factors are at an increased risk for developing dementia, is beneficial in developing both screening and treatment or prevention plans for dementia.

Similar diagnostic screens for insulin resistance have been developed by the World Health Organization and the National Cholesterol Education Program's Adult Treatment Panel III (ATP III). There are several labels for this syndrome which, for purposes of this application, all refer to the same underlying condition of insulin resistance. These include “Metabolic Syndrome”, “Dysmetabolic Syndrome X”, and “Syndrome X”.

Although the risk of some of the physiological conditions that are part of metabolic syndrome for diseases such as heart disease and stroke has been previously understood and described, the present invention is the first to suggest a connection between metabolic syndrome and dementia, and to suggest screening, prevention, and treatment methods based on this connection. Furthermore, sleep and mood disorders accompanying insulin resistance can aggravate symptoms of dementia.

These risk factors are related to several biochemical pathways. Firstly, there exists an enzyme known as insulin-degrading enzyme (IDE). IDE is an about 110-kDa thiol zinc metalloendopeptidase located in the cytosol, in peroxisomes, and on the cell surface (W. Farris et al., “Insulin-Degrading Enzyme Regulates the Levels of Insulin, Amyloid β-Protein, and the β-Amyloid Precursor Protein Intracellular Domain In Vivo,” Proc. Natl. Acad. Sci. USA 100: 4162-4167 (2003)). IDE cleaves small proteins of diverse sequence, many of which share a propensity to form β-pleated sheet-rich amyloid fibrils under certain conditions. These proteins include amyloid β-protein (Aβ), insulin, glucagons, amylin, atrial natriuretic factor, and calcitonin. The present invention is based on the idea that the increased insulin present in insulin resistance or metabolic syndrome competes with β-amyloid for IDE. The more insulin is present, the slower is the degradation of β-amyloid by IDE. This causes an excessive buildup of β-amyloid, which is one of the factors believed associated with the development of dementia, particularly Alzheimer's disease.

Secondly, the presence of excess insulin also increases the number of N-methyl-D-aspartate (NMDA) receptors in the nervous system, and increases the activity of these NMDA receptors in generating ionic currents (G.-Y. Liao & J. P. Leonard, “Insulin Modulation of Cloned Mouse NMDA Receptor Currents in Xenopus Oocytes,” J. Neurochem. 73: 1510-1519 (1999); V. A. Skeberdis et al., “Insulin Promotes Rapid Delivery of N-Methyl-D-Aspartate Receptors to the Cell Surface by Exocytosis,” Proc. Natl. Acad. Sci. USA 98: 3561-3566 (2001); J. M. Christie et al., “Insulin Causes a Transient Tyrosine Phosphorylation of NR2A and NR2B Receptor Subunits in Rat Hippocampus,” J. Neurochem. 72: 1523-1528(1999)). These NMDA receptors also respond to β-amyloid, which possibly indirectly causes further activation of the receptors as well as having its own spectrum of neurotoxic activities. In fact, β-amyloid is believed to cause apoptotic cell death through the generation of nitric oxide (NO) and other free radicals (W.-D. Lee et al., “Cell Death Induced by β-Amyloid 1-40 in MES 23.5 Hybrid Clone: The Role of Nitric Oxide and NMDA-Gated Channel Activation Leading to Apoptosis,” Brain Res. 686: 49-50 (1995)). The activation of NO synthesis appears to be mediated by calcium ions entering through activated NMDA-gated channels. There are several potential links between excitotoxic (NMDA receptor-mediated) damage and the primary insults of Alzheimer's disease, which, based on rare familial forms of the disease, are believed to involve toxicity from misfolded mutant proteins (recently reviewed by Rogawski M A and Wenk G L. The neuropharmacological basis for the use of memantine in the treatment of Alzheimer's disease. CNS Drug Rev 2003; 9:275-308)>These proteins include fibrillar β-amyloid peptide (Aβ) and hyperphosphorylated tau proteins (Selkoe D J. Alzheimer's disease: genes, proteins, and therapy. Physiol Rev 2001; 81:741-766). For example, oxidative stress and increased intracellular Ca²⁺ generated by Aβ have been reported to enhance glutamate-mediated neurotoxicity in vitro. Additional experiments suggest that Aβ can increase NMDA responses and thus excitotoxicity (Wu J, Anwyl R and Rowan M I beta-Amyloid-(1-40) increases long-term potentiation in rat hippocampus in vitro. Eur J Pharmacol 1995; 284:R1-3; Mattson M P, Cheng B, Davis D, Bryant K, Lieberburg I and Rydel R E. beta-Amyloid peptides destabilize calcium homeostasis and render human cortical neurons vulnerable to excitotoxicity. J Neurosci 1992; 12:376-389; Koh J Y, Yang L L and Cotman C W. Beta-amyloid protein increases the vulnerability of cultured cortical neurons to excitotoxic damage. Brain Res 1990; 533:315-320). Another potential link comes from recent evidence that glutamate transporters are down regulated in Alzheimer's disease and that Aβ can inhibit glutamate reuptake or even enhance its release (Topper R, Gehrmann J, Banati R, Schwarz M, Block F, Noth J and Kreutzberg G W. Rapid appearance of beta-amyloid precursor protein immunoreactivity in glial cells following excitotoxic brain injury. Acta Neuropathol (Berl) 1995; 89:23-28; Harkany T, Abraham I, Timmerman W, Laskay G, Toth B, Sasvari M, Konya C, Sebens J B, Korf J, Nyakas C, Zarandi M, Soos K, Penke B and Luiten P G. beta-amyloid neurotoxicity is mediated by a glutamate-triggered excitotoxic cascade in rat nucleus basalis. Eur J Neurosci 2000; 12:2735-2745). Finally, excessive NMDA receptor activity has been reported to increase the hyperphosphorylation of tau, which contributes to neurofibrillary tangles (Couratier P, Lesort M, Sindou P, Esclaire F, Yardin C and Hugon J. Modifications of neuronal phosphorylated tau immunoreactivity induced by NMDA toxicity. Mol Chem Neuropathol 1996; 27:259-273). The NMDA receptor antagonist memantine has been found to offer protection from intrahippocampal injection of Aβ (Miguel-Hidalgo J J, Alvarez X A, Cacabelos R and Quack G. Neuroprotection by memantine against neurodegeneration induced by beta-amyloid(1-40). Brain Res 2002; 958:210-221). Moreover, memantine improved performance on behavioral tests (T-maze and Morris water maze) in a transgenic mouse model of Alzheimer's disease consisting of a mutant form of amyloid precursor protein and presenilin 1 (Tania H, Minkevicine R and Banjeree P. Behavioral effects of subchronic memantine treatment in APP/PS1 double mutant mice modeling Alzheimer's disease. J Neurochem 2003; 85(Suppl 1):42). Additionally, memantine was recently found to reduce tau hyperphosphorylation, at least in culture (Iqbal K, Li L, Sengupta A and Grundke-Iqbal I. Memantine restores okadaic acid-induced changes in protein phosphatase-2A, CAMKII and tau hyperphosphorylation in rat. J Neurochem 2003; 85(Suppl 1):42).

It is known that NMDA receptors are linked to learning and memory processes. However, excessive prolonged activation of NMDA receptors leads to excessive Ca²⁺ influx, which, in turn, is neurotoxic (Lipton and Rosenberg, ibid.). It has been demonstrated, at least in hippocampal slices, that if NMDA receptors are overstimulated due to Mg²⁺ removal or application of an exogenous NMDA agonist, neuronal plasticity such as long-term potentiation (LTP), a critical element in the current neuronal model of memory formation, is impaired and neurons may be injured.

With respect to screening for susceptibility to dementia, in general, a screening method according to the present invention comprises:
(1) screening a patient for one or more of the following indications:
   (a) a waistline of 40 inches or greater for men or 35 inches or greater for women as measured across the belly;
   (b) a body mass index greater than 25 kg/m²
   (c) a blood pressure of 130/85 mm Hg or more;
   (d) a triglyceride level of above 150 mg/deciliter;
   (e) a fasting blood glucose level greater than about 100 mg/dl;
   (f) a blood glucose level greater than 140 mg/dl measured 2 hours after a 75 gram oral administration of glucose;
   (g) a high density lipoprotein (HDL) level less than 40 mg/dl for men or less than 50 mg/dl for women; and
   (h) C-reactive protein-high sensitivity (CRP-hs);
(2) determining how many of the indications are present in the patient; and
(3) correlating the number of indications with the risk of developing dementia, such that the presence of at least one indication indicates an increased risk of developing dementia and the presence of at least three indications indicates a substantially increased risk of developing dementia.

Preferably, the screening method of the present invention is coupled with suitable modes of intervention, such as weight control methods and exercise programs, to eliminate one or more of the indications and thereby to reduce the risk of developing dementia.

With respect to the treatment or prevention of dementia, the present invention encompasses three embodiments for the treatment or prevention of dementia:
(1) administering an agent that minimizes insulin resistance, thereby preventing excess biosynthesis of insulin, in a quantity sufficient to minimize insulin resistance;
(2) administering an agent that modulates the activity of IDE such that insulin competes less efficiently with β-amyloid protein for the IDE, in a quantity sufficient to modulate the activity of IDE; or
(3) administering an agent that blocks the consequences of NMDA receptor activation, such as by minimizing the generation of NO and other harmful free radicals, in a quantity sufficient to block the consequences of NMDA receptor activation.

Alternatively, treatment to prevent dementia includes administering an agent of 1, 2 or 3, above, along with an agent that treats sleep and mood or other secondary effectors.

These methods can be used for the prevention of dementia in a patient identified by the screening test described above as being at risk for dementia in conjunction with that screening test. Alternatively, these methods can be used for the treatment of dementia in a patient already diagnosed with dementia.

As used herein, the term “co-treatment” means treatment of risk factors for insulin resistance and secondary effectors for the prevention and treatment of dementia.

As used herein the term “secondary effectors” means factors that aggravate neurological degenerative disorders, for example, sleep and mood disorders.

As used herein, the term “treatment” encompasses any result that indicates either stabilization of the condition or improvement in one or more indicators of cognitive functioning or emotional stability, but does not require or demand a complete cure.

In the first of these embodiments, the insulin resistance can be minimized by treatment with at least one agent that either upregulates the catabolism of glucose and other carbohydrates or downregulates the biosynthesis of lipids.

These agents include, but are not limited to, the following: (1) insulin (rapid, short-acting, intermediate, long-acting, or inhaled); (2) sulfonylureas, including tolbutamide, acetohexamide, tolazamide, chlorpropamide, glyburide, glipizide, and gliclazide, as well as their analogues; (3) meglitinides and their analogues; (4) biguanides, including metformin, and their analogues; (5) thiazolidinediones, including rosiglitazone and pioglitazone, and their analogues; (5) α-glucosidase inhibitors, including acarbose and their analogues; (6) orlistat (Xenical) and other pancreatic lipase inhibitors and their analogues; (7) IGF-1 and IGF-1 analogues; (8) pigment epithelium derived factor (PEDF) and its analogues; (9) glycogen synthase kinase-3β inhibitors and their analogues; (10) ghrelin obesity drugs and related compounds and analogues; (11) 5-hydroxytryptamine (serotonin)-related molecules and analogues; (12) β₃-adrenergic agonists, including phenoxybenzamide, and their analogues; (13) leptin, leptin agonists, and their analogues; (14) melanocortin 4 agonists and their analogues; (15) Retinoid X Receptor modulators and their analogues; (16) adiponectin receptor agonists and their analogues; (17) modulators of glucocorticoid receptors and their analogues; (18) thyromimetics and other agonists for thyroid hormone receptors, as well as their analogues; (19) peroxisome proliferator activated receptor modulators, including fibrate drugs, fatty acids (clofibric acid, fenofibrate, etiofibrate, gemfibrozil), and prostaglandin derivatives, as well as analogues of these agents; (20) retinoic acid receptor modulators and their analogues; (21) estrogen receptor agonists and their analogues; (22) androgen receptor modulators and their analogues; (23) progesterone receptor modulators and their analogues; (24) mineralocorticoid receptor modulators and their analogues; (25) insulin secretagogues and their analogues; (26) insulin analogues and mimetics, and analogues of such compounds; (27) insulin receptor agonists and their analogues; (28) helix-loop-helix transcription factors such as SREBP-like factors and ADD1 and their analogues; (29) CAAT/enhancer binding protein modulators and their analogues; (30) AP-1 like factors including protein kinase C and protein kinase A; (31); growth hormones and their agonists and antagonists; (32) tumor necrosis factor and related compounds; (33) cytokines, including IL-1 and TGF-β; (34) non-steroidal anti-inflammatory drugs and their analogues; (35) prostacyclins and their analogues; (36) dihydroepiandrosterone and its analogues; (37) fetuin; (38) amylin modulators and their analogues; (39) prolactin; (40) niacin, acepimox, and other nicotinic acid derivatives and their analogues; (41); triacsins and their analogues; (42) amphetamines and their analogues and derivatives; (43) endorphin agonists and their analogues; (44) somatostatin; (45) cholecystokinin; (46) bombesin; (47) gastrin; (48); corticotrophin-releasing hormone (CRH) and its analogues; (49) adrenocorticotropic hormone (ACTH) a and b and their analogues; (50) α-melanocyte stimulating hounone (MSH) and its analogues; (51) gastric inhibitory peptides; (52) agents that lower plasma cortisol either via synthesis of cortisol or via cortisol inhibition; and (53) compounds acting through Insulin-Like Growth Factor. However, of these agents, insulin (category (1)) and the sulfonylureas, including tolbutamide, acetohexamide, tolazamide, chlorpropamide, glyburide, glipizide, and gliclazide, as well as their analogues (category (2)) should only be administered to patients who have already developed diabetes with hyperglycemia.

Other agents suitable for use in methods according to the present invention are disclosed in U.S. Pat. No. 6,068,976 to Briggs et al., incorporated by this reference.

Still other agents are suitable for use in methods according to the present invention to minimize insulin resistance. These include GLP 1 (glucagons like peptide), DPP IV inhibitors (diethyly peptidase inhibitors), INGAP (islet neogenesis associated protein), statins, angiotensin converting enzyme (ACE) inhibitors), angiotensin receptor blockers (ARBs), bromocriptinekabergoline, and colesevelam.

As used herein, the term “agonist” refers to a compound that binds specifically to a receptor and potentiates the action normally carried out by that receptor, such as intracellular or intercellular signaling.

As used herein, the term “antagonist” refers to a compound that binds specifically to a receptor and blocks or inhibits the action normally carried out by that receptor.

As used herein, the term “modulator” refers to both agonists and antagonists.

As used herein, the term “analogue” refers to a compound having a structural relationship with the named compound and a substantially similar activity, including homologues that differ by one or more carbon atoms and isosteres.

In addition, prodrugs and salt forms of these compounds are encompassed by the present invention. It is well known that organic compounds; including compounds having activities suitable for methods according to the present invention, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound or analogue includes such salt forms as occur at physiological pH or at the pH of a pharmaceutical composition unless specifically excluded.

Similarly, prodrug esters can be formed by reaction of either a carboxyl or a hydroxyl group on compounds or analogues suitable for methods according to the present invention with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art.

In addition, where compounds recited above are optically active, both the optically active form and the racemic mixture are encompassed by the present invention unless the racemic mixture is specifically excluded.

Additionally, where the compounds recited above include peptides or proteins, variants of those molecules having conservative amino acid substitutions are included. It is a well-established principle of protein and peptide chemistry that certain amino acids substitutions, entitled “conservative” amino acid substitutions, can frequently be made in a protein or a peptide without altering either the confirmation or the function of the protein or peptide. Such changes include substituting any of isoleucine (I), valine (V), and leucine (L) for any other of these amino acids; aspartic acid (D) for glutamic acid (E) and vice versa; glutamine (Q) for asparagine (N) and vice versa; and serine (S) for threonine (T) and vice versa. The above-mentioned substitutions are not the only amino acid substitutions that can be considered “conservative.” Other substitutions can also be considered conservative, depending on the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

In the second of these embodiments, the activity of IDE can be modulated by either reducing the synthesis of insulin or modulating the activity of IDE so that it more efficiently degrades β-amyloid.

The following agents can reduce the synthesis of insulin: (1) thiazolidinediones, including rosiglitazoneand pioglitazone; and (2) somatostatin. In addition, any agent that improves insulin sensitivity will lead to a decrease in insulin production. All the of these agents work indirectly, not directly on the beta cell.

Insulin and β-amyloid are believed to compete for the active site of DE, so anything that reduces the level of insulin can allow more effective degradation of β-amyloid.

In the third embodiment, the neurotoxicity and inhibition of long-term potentiation can be decreased or minimized either by administration of an agent that prevents the consequences of free radical release, particularly the release of NO free radicals, or that inhibits the activation of NMDA receptors.

Agents that prevent the consequences of free radical release, particularly the release of NO free radicals, include: (1) nitroglycerin in various forms, including to tablets and spray; (2) isosorbide; (3) amyl nitrate; and (4) sodium nitroprusside.

Agents that inhibit the activation of NMDA receptors include: (1) dizocilpine and its analogues; (2) cerestat and its analogues; (3) amantadine and its derivatives, including amantadine (1-adamantanamine hydrochloride), memantine (1-amino-3,5-dimethyladamantine), and rimantadine (α-methyl-1-adamantanemethylamine hydrochloride), as well as other substituted amantadine derivatives, including 1-acetamido-3,5-dimethyl-7-hydroxyadamantane; 1-amino-3,5-dimethyl-7-hydroxyadamantane hydrochloride; 1-t-butylcarbamate-3,5-dimethyl-7-hydroxyadamantane; 1-t-butylcarbamate-3,5-dimethyl-7-nitrateadamantane; 1-amino-3,5-dimethyl-7-nitrateadamantane hydrochloride; 1-acetamido-3,5-dimethyl-7-nitrateadamantane; 1,1-dibenzylamino-3,5-dimethyl-7-hydroxyadamantane; 1-amino-3,5-dimethyl-7-acetoxyadamantane hydrochloride; 1-(benzyloxycarbonyl)amino-3,5-dimethyl-7-hydroxyadamantane; 1-(benzyloxycarbonypamino-3,5-dimethyl-7-(3-bromopropylcarbonyloxy)adamantine; 1-(benzxyloxycarbonyl)amino-3,5-dimethyl-7(3-nitratepropylcarbonyloxy)adarnantine; 1-acetamido-3,5-dimethyl-7-carboxylic acidadamantane; 1-acetamido-3,5-dimethyl-7-hydroxymethyladamantane; 1-amino-3,5-dimethyl-7-hydroxymethyladamantane hydrochloride; 1-(benzyloxycarbonyl)arnino-3,5-dimethyl-7-hydroxyrmethyladamantane; 1-(benzyloxycarbonyl)amino-3,5-dimethyl-7-nitratemethyladamantane; 1-amino-3,5-dimethyl-7-nitratemethyladamantane hydrobromide; and 1-acetamido-3,5-dimethyl-7-nitratemethyladamantane, as well as other substituted adamantane derivatives. Such compounds are disclosed in U.S. Pat. No. 6,620,845 to Wang et al. and in U.S. Pat. No. 5,334,618 to Lipton, both incorporated herein by this reference.

The particular compounds or agents useful in methods according to the present invention can be administered to a patient either by themselves or in pharmaceutical compositions where it is mixed with suitable carriers or excipient(s). In treating a patient suffering from or at risk of dementia, a therapeutically effective amount of an agent or agents as described above is administered. A therapeutically effective dose refers to that amount of the compound that results in amelioration of symptoms or a prolongation of survival in a patient.

The compounds also can be prepared as pharmaceutically acceptable salts. Examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. (See e.g., PCT Patent Application No. PCT/US92/03736, incorporated herein by this reference). Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols and physiologically compatible solvents.

In addition, the molecules tested can be used to determine the structural features that enable them to act on the appropriate step of the pathways disclosed herein, including insulin synthesis and resistance, the activity of IDE, the activity of NMDA receptors, and free radical generation, especially NO generation as the result of β-amyloid stimulation of NMDA receptors, and thus to select molecules useful in this invention. Those skilled in the art will know how to design drugs from lead molecules, using techniques such as those disclosed in PCT Publication No. WO 94/18959, incorporated by reference herein.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human patients. The dosage of such compounds lies preferably within a range of circulating concentrations that include the ED₅₀ with little or no toxicity. The dosage may vary within this range depending upon the dosage form employed and the route of administration utilized.

For any compound used in methods according to the present invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal effect in the particular step of the reaction affected). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et a., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of dementia will vary with the severity of the dementia and with the route of administration. The severity of the dementia may, for example, be evaluated, in part, by standard prognostic evaluation methods for assessing cognitive and other mental function, such as scales for evaluating these functions. Further, the dose and perhaps dose frequency, will also vary according to the age, body weight, and response of the individual patient, as well as other conditions affecting pharmacodynamic parameters such as liver and kidney function.

Depending on the severity of the dementia being treated, such agents may be formulated and administered systemically or locally. Techniques for formulation and administration may be found in Remington's Pharmaceutical Sciences, 19th ed., Mack Publishing Co., Easton, Pa. (1995). Tablets, capsules, pills, powders, granules, dragees, gels, slurries, ointments, solutions, suppositories, injectable formulations, inhalants, and aerosols are examples of such formulations. Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, intrratracheal, or intraocular injections, just to name a few.

For injection, the agents useful in methods according to the present invention can be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions useful in methods according to the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in methods according to the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions suitable for use in methods according to the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Agents suitable for use in methods according to the present invention can also be delivered in an aerosol spray preparation from a pressurized pack, a nebulizer or from a dry powder inhaler. Suitable propellants that can be used in a nebulizer include, for example, dichlorodifluoromethane, trichlorofluoromethane, dichlorotetrafluoroethane and carbon dioxide. The dosage can be determined by providing a valve to deliver a regulated amount of the compound in the case of a pressurized aerosol.

Other methods of delivery can be used.

Methods according to their present invention can be used for the treatment or prevention of a number of conditions marked by neuronal degeneration, including, but not limited to, dementia, including the dementia associated with Alzheimer's disease, vascular dementia, Huntington's disease, amyotrophic lateral sclerosis (Lou Gehrig's disease), dementia associated with AIDS (HIV-associated dementia), glaucoma depression, drug dependence/tolerance/addiction, neurolathyrism (resulting from ingestion of β-N-oxalylamino-L-alanine found in chickpeas), “Guam disease” (resulting from ingestion of β-N-methyl-amino-L-alanine found in flour from cycad seeds), olivo-pontocerebellar atrophy, MELAS syndrome (mitochondrial myopathy, encephalopathy, lactic acidosis, and stroke-like episodes), Rett syndrome, homocysteinuria, hyperprolinemia, hyperglycinemia (non-ketotic), hepatic encephalopathy, uremic encephalopathy, 4-hydroxybutyric aciduria, trauma to the central nervous system, carbon monoxide poisoning, lead poisoning, and domoic acid poisoning (a glutamate-like agonist found in contaminated shellfish, especially mussels). However, methods according to the present invention are particularly suitable for the treatment or prevention of the dementia of Alzheimer's disease.

Methods according to their present invention further include the treatment of dementia by administering to a patient an agent that minimizes insulin resistance and an agent treating a secondary effector, thereby preventing excess biosynthesis of insulin, in a quantity sufficient to minimize insulin resistance, wherein the secondary effector is sleep, mood, or sleep and mood. This is a co-treatment.

Agents used in the treatment of sleep and mood disorder include, but are not limited to, (1) benzodiazepines, including buspirone and zolpidem; (2) carbamates, including, meprobamate; (3) barbiturates, including phenobarbital and secobarbital; (4) phenothiazines, including chlorpromazine and mesoridazine; (5) butyrophenones, including haloperidol; (6) other heterocyclic compounds, including clozapine and olanzapine; (7) lithium and clonazepam; (8) monoamine oxidase inhibitors, including phenelzine; (9) tricyclics, including amitriptyline and imipramine; (10) selective serotonin reuptake inhibitors, including fluoxetine and paroxetine; and (11) heterocyclic second- and third-generation antidepressants, including mirtazapine and venlafaxine. Agents used for the treatment of sleep and mood disorder may be found in Goodman and Gilman's: The Pharmacological Basis of Therapeutics, 10^{th} ed., The McGraw-Hill Companies, Inc., 2001, Ch. 17, 19-20. These treatments of sleep and mood disorder are known to an ordinary person in the pertinent prior art.

Although the present invention has been described in considerable detail with regard to certain preferred versions thereof, other versions are possible. Therefore, the spirit and scope of the appended claims should not be limited to the descriptions of the preferred versions contained herein.

1. A method for screening a patient for susceptibility to dementia comprising the steps of:
(a) screening a patient for one or more of the following indications:
(i) a waistline of 40 inches or greater for men or 35 inches or greater for women as measured across the belly,
(ii) a body mass index greater than 25 kg/m²
(iii) a blood pressure of 130/85 mm Hg or more;
(iv) a triglyceride level of above 150 mg/deciliter;
(v) a fasting blood glucose level greater than about 100 mg/dl;
(vi) a blood glucose level greater than 140 mg/dl measured 2 hours after a 75 gram oral administration of glucose; and
(vii) a high density lipoprotein (HDL) level less than 40 mg/dl for men or less than 50 mg/dl for women;
(2) determining how many of the indications are present in the patient; and
(3) correlating the number of indications with the risk of developing dementia, such that the presence of at least one indication indicates an increased risk of developing dementia and the presence of at least three indications indicates a substantially increased risk of developing dementia.

2. A method for the prevention of dementia comprising:
(a) identifying a patient at risk for susceptibility to dementia by:
(i) screening a patient for one or more of the following indications:
(A) a waistline of 40 inches or greater for men or 35 inches or greater for women as measured across the belly;
(B) a body mass index greater than 25 kg/m²
(C) a blood pressure of 130/85 mm Hg or more;
(D) a triglyceride level of above 150 mg/deciliter;
(E) a fasting blood glucose level greater than about 100 mg/dl;
(F) a blood glucose level greater than 140 mg/dl measured 2 hours after a 75 gram oral administration of glucose; and
(G) a high density lipoprotein (HDL) level less than 40 mg/dl for men or less than 50 mg/dl for women;
(ii) determining how many of the indications are present in the patient; and
(iii) correlating the number of indications with the risk of developing dementia, such that the presence of at least one indication indicates an increased risk of developing dementia and the presence of at least three indications indicates a substantially increased risk of developing dementia; and
(b) administering an agent that minimizes insulin resistance, thereby preventing excess biosynthesis of insulin, in a quantity sufficient to minimize insulin resistance thereby reducing sleep and mood disorders, so that the risk of developing dementia is reduced.

3. The method of claim 2 wherein the agent that minimizes insulin resistance is selected from the group consisting of: (1) insulin; (2) sulfonylureas and their analogues; (3) meglitinides and their analogues; (4) biguanides and their analogues; (5) thiazolidinediones and their analogues; (5) α-glucosidase inhibitors; (6) pancreatic lipase inhibitors and their analogues; (7) IGF-1 and IGF-1 analogues; (8) pigment epithelium derived factor (PEDF) and its analogues; (9) glycogen synthase kinase-3β inhibitors and their analogues; (10) ghrelin obesity drugs and related compounds and analogues; (11) 5-hydroxytryptamine (serotonin)-related molecules and analogues; (12) β₃-adrenergic agonists and their analogues; (13) leptin, leptin agonists, and their analogues; (14) melanocortin 4 agonists and their analogues; (15) Retinoid X Receptor modulators and their analogues; (16) adiponectin receptor agonists and their analogues; (17) modulators of glucocorticoid receptors and their analogues; (18) thyromimetics and other agonists for thyroid hormone receptors, and their analogues; (19) peroxisome proliferator activated receptor modulators and prostaglandin derivatives, and analogues of peroxisome proliferators receptor modulators and prostaglandin derivatives; (20) retinoic acid receptor modulators and their analogues; (21) estrogen receptor agonists and their analogues; (22) androgen receptor modulators and their analogues; (23) progesterone receptor modulators and their analogues; (24) mineralocorticoid receptor modulators and their analogues; (25) insulin secretagogues and their analogues; (26) insulin analogues and mimetics, and analogues of insulin analogues and mimetics; (27) insulin receptor agonists and their analogues; (28) helix-loop-helix transcription factors and their analogues; (29) CAAT/enhancer binding protein modulators and their analogues; (30) AP-1 like factors; (31); growth hormones and their agonists and antagonists; (32) tumor necrosis factor and related compounds; (33) cytokines; (34) non-steroidal anti-inflammatory drugs and their analogues; (35) prostacyclins and their analogues; (36) dihydroepiandrosterone and its analogues; (37) fetuin; (38) amylin modulators and their analogues; (39) prolactin; (40) niacin, acepimox, and other nicotinic acid derivatives and their analogues; (41); triacsins and their analogues; (42) amphetamines and their analogues and derivatives; (43) endorphin agonists and their analogues; (44) somatostatin; (45) cholecystokinin; (46) bombesin; (47) gastrin; (48); corticotrophin-releasing hormone (CRH) and its analogues; (49) adrenocorticotropic hormone (ACTH) a and b and their analogues; (50) α-melanocyte stimulating hormone (MSH) and its analogues; (51) gastric inhibitory peptides; (52) agents that lower plasma cortisol either via synthesis of cortisol or via cortisol inhibition; and (53) compounds acting through Insulin-Like Growth Factor.

4. The method of claim 3 wherein the agent that minimizes insulin resistance is insulin.

5. The method of claim 4 wherein the insulin is administered in a form selected from the group consisting of rapid, short-acting, intermediate, long-acting, and inhaled insulin.

6-44. (canceled)

45. A method for the treatment of dementia comprising the step of administering to a patient diagnosed with dementia an agent that minimizes insulin resistance, thereby preventing excess biosynthesis of insulin, in a quantity sufficient to minimize insulin resistance, to treat the dementia.

46. The method of claim 45 wherein the agent that minimizes insulin resistance is selected from the group consisting of; (1) insulin; (2) sulfonylureas and their analogues; (3) meglitinides and their analogues; (4) biguanides and their analogues; (5) thiazolidinediones and their analogues; (5) a-glucosidase inhibitors; (6) pancreatic lipase inhibitors and their analogues; (7) IGF-1 and IGF-1 analogues; (8) pigment epithelium derived factor (PEDF) and its analogues; (9) glycogen synthase kinase-3β inhibitors and their analogues; (10) ghrelin obesity drugs and related compounds and analogues; (11) 5-hydroxytyptamine (serotonin)-related molecules and analogues; (12) β₃ -adrenergic agonists and their analogues; (13) leptin, leptin agonists, and their analogues; (14) melanocortin 4 agonists and their analogues; (15) Retinoid X Receptor modulators and their analogues; (16) adiponectin receptor agonists and their analogues; (17) modulators of glucocorticoid receptors and their analogues; (18) thyromimetics and other agonists for thyroid hormone receptors, and their analogues; (19) peroxisome proliferator activated receptor modulators and prostaglandin derivatives, and analogues of peroxisome proliferators receptor modulators and prostaglandin derivatives; (20) retinoic acid receptor modulators and their analogues; (21) estrogen receptor agonists and their analogues; (22) androgen receptor modulators and their analogues; (23) progesterone receptor modulators and their analogues; (24) mineralocorticoid receptor modulators and their analogues; (25) insulin secretagogues and their analogues; (26) insulin analogues and mimetics, and analogues of insulin analogues and mimetics; (27) insulin receptor agonists and their analogues; (28) helix-loop-helix transcription factors and their analogues; (29) CAAT/enhancer binding protein modulators and their analogues; (30) AP-1 like factors; (31); growth hormones and their agonists and antagonists; (32) tumor necrosis factor and related compounds; (33) cytokines; (34) non-steroidal anti-inflammatory drugs and their analogues; (35) prostacyclins and their analogues; (36) dihydroepiandrosterone and its analogues; (37) fetuin; (38) amylin modulators and their analogues; (39) prolactin; (40) niacin, acepimox, and other nicotinic acid derivatives and their analogues; (41); triacsins and their analogues; (42) amphetamines and their analogues and derivatives; (43) endorphin agonists and their analogues; (44) somatostatin; (45) cholecystokinin; (46) bombesin; (47) gastrin; (48); corticotrophin-releasing hormone (CRH) and its analogues; (49) adrenocorticotropic hormone (ACTH) a and b and their analogues; (50) a-melanocyte stimulating hormone (MSH) and its analogues; (51) gastric inhibitory peptides; (52) agents that lower plasma cortisol either via synthesis of cortisol or via cortisol inhibition; and (53) compounds acting through Insulin-Like Growth Factor

47. The method of claim 46 wherein the agent that minimizes insulin resistance is insulin.

48. The method of claim 47 wherein the insulin is administered in a form selected from the group consisting of rapid, short-acting, intermediate, long-acting, and inhaled insulin.

49-86. (canceled)

87. A method for the treatment of dementia comprising the step of administering to a patient diagnosed with dementia: (1) an agent that minimizes insulin resistance; and (2) an agent treating a secondary effector.

88. The method of claim 87 wherein the secondary effector is sleep.

89. The method of claim 87 wherein the secondary effector is mood.

90. The method of claim 87 wherein the secondary effector is sleep and mood.
